(19) United States
(12) Patent Application Publication
(10) Pub. No.: US 2009/0029904 A1
(43) Pub. Date: Jan. 29, 2009

(54) Compositions and methods for treatment of insulin-resistance diseases

(76) Inventor: Sean Oldham, La Jolla, CA (US)

Correspondence Address: Biotechnology Law Group;c/o Portfolioip, P.O. Box 52050, Minneapolis, MN, 55402, US

(21) Appl. No.: 11/880,350

(22) Filed: Jul. 20, 2007

Related U.S. Application Data
(60) Provisional application No. 60/832,311, filed on Jul. 21, 2006.

(51) Int. Cl. A61K 38/28; A61K 31/155; C12Q 1/68; A61P 3/00; A61P 3/04; A61P 3/10; A61P 9/00

(52) U.S. Cl. 514/4; 514/635; 435/6

(57) ABSTRACT

Compositions and methods are provided for modulation of Target of Rapamycin (TOR) activity. Reduction of TOR blocks insulin resistance and metabolic syndrome phenotypes caused by expression of a constitutively activated version of the insulin responsive transcription factor, FOXO. These TOR-mediated responses are also linked with protection against age-dependent functional organ senescence, e.g., heart decline, as well as increased longevity without changes in resistance to starvation and oxidative stresses.

### STATEMENT OF GOVERNMENT RIGHTS

The invention was supported, at least in part, by a grant from the Government of the United States of America (grant no. HL84949 from the National Institutes of Health). The Government has certain rights to the invention.

### BACKGROUND

1. Technical Field

The present invention relates to materials and methods for treatment of metabolic syndrome, insulin-resistance diseases like obesity and Type II diabetes, and aging and organ senescence.

2. Background of the Invention

Metabolic syndrome is a cluster of metabolic abnormalities like elevated glucose and lipid levels related to a state of insulin resistance. The major cause of metabolic syndrome and diabetes in humans is a reduction of insulin signaling, but the underlying pathways and mechanisms are not completely understood. Likewise, excessive nutrients can lead to nutrient toxicity and the metabolic syndrome. Thus, dysregulation of energy homeostasis can lead to metabolic disturbances and predisposition to a variety of endocrine diseases including diabetes, cardiovascular disease, and cancer (Biddinger and Kahn, Ann. Rev. Physiol. 68:1-36, 2006; Kahn et al., Cell Metab. 1:15-25, 2005; Kitamura et al., Ann. Rev. Physiol. 65:313-332, 2003; Lee and White, Arch. Pharm. Res. 27:361-370, 2004).

One major system that regulates energy homeostasis in higher metazoa is the insulin/IGF pathway. The functionally conserved components of the insulin/IGF pathway, like insulin, the insulin receptor (InR), insulin receptor substrate (IRS), phosphoinositide 3-kinase (PI3K), protein kinase B (PKB, also known as Akt) and the forkhead transcription factor FOXO, have been shown to be involved in glucose and lipid homeostasis (Barthel et al., Trends Endocrin. Metab. 16:183-189, 2005; Biddinger and Kahn, Ann. Rev. Physiol. 68:1-36, 2006; Kahn et al., Cell Metab. 1:15-25, 2005; Kitamura et al., Ann. Rev. Physiol. 65:313-332, 2003; Lantz and Kaestner, Clin. Sci. 108:195-204, 2005; Lee and White, Arch. Pharm. Res. 27:361-370, 2004) as well as growth and aging (Accili and Arden, Cell 117:421-426, 2004; Burgering and Kops, Trends Biochem. Sci. 27:352-360, 2002; Finch and Ruvkun, Ann. Rev. Genomics Human Genet. 2:435-462, 2001; Greer and Brunet, Oncogene 24:7410-7425, 2005; Kenyon, Cell 120:449-460, 2005; Tran et al., Sci. STKE 2003, RE5, 2003). Loss of insulin signaling in the periphery and in pancreatic beta cells can lead to hyperglycemia and diabetes (Kahn, Diabetologia 46:3-19, 2003; Nandi et al., Physiol. Rev. 84:623-647, 2004; Rhodes and White, Eur. J. Clin. Invest. 32 (Suppl 3):3-13, 2002). For example, disruption of the IGFR gene in the whole animal reduces islet size and insulin secretion (Efstratiadis, Intl. J. Dev. Biol. 42:955-976, 1998; Nakae et al., Endocr. Rev. 22:818-835, 2001). IRS1 knock-out mice are hyperglycemic, but their pancreatic beta cells hypertrophy to compensate for increased peripheral insulin resistance (Araki et al., Nature 372:186-190, 1994; Burks and White, Diabetes 50 [Supp. 1]:S140-145, 2001). In contrast, JRS2 knock-out mice are diabetic because their pancreatic beta cells are absent due to increased cell death (Burks and White, Diabetes 50 [Supp. 1]:S140-145, 2001). Additionally, systemic loss of insulin signaling in metazoans leads to elevated lipids as seen in the Daf-2 mutant worms, Chico/IRS mutant flies, and IRS2 ablated mice (Böhni et al., Cell 97:865-875, 1999; Burks et al., Nature 407:377-382, 2000; Kenyon et al., Nature 366:461-464, 1993; Kimura et al., Science 277:942-946, 1997).

Many of these insulin/IGF-mediated metabolic effects depend on the winged helix transcription factor, FOXO. FOXO was first identified in the worm, Caenorhabdatis elegans as Daf-16, a mutation that can suppress the increased lipid levels and longevity caused by loss of Daf-2, the worm InR ortholog (Lin et al., Science 278:1319-1322, 1997; Ogg et al., Nature 389:994-999, 1997). There is a single evolutionarily conserved FOXO ortholog present in the Drosophila genome (Junger et al., J. Biol. 2:20, 2003; Kramer et al., BMC Dev. Biol. 3:5, 2003; Puig et al., Genes Dev. 17:2006-2020, 2003). There are three mammalian FOXO genes (FOXO1, FOXO3a, and FOXO4). FOXO1 genetically interacts with the insulin pathway to control glucose homeostasis in both peripheral tissues and pancreatic beta cells (Accili and Arden, Cell 117:421-426, 2004). Constitutively activated FOXO1 (resistant to insulin/IGF-mediated inactivation) in liver and pancreatic beta cells causes hepatic insulin resistance and loss of pancreatic beta cells via increased apoptosis and loss of compensation due to loss of PDX1, whereas reduction of FOXO1 function can reverse the loss of pancreatic beta cells and hyperglycemia seen in the IRS2 ablated mice (Kitamura et al., J. Clin. Investigation 110:1839-1847, 2002; Nakae et al., Nature Genet. 32:245-253, 2002). Thus, FOXO is an important regulator of insulin signaling in insulin sending and receiving tissues and has many critical functions in mediating glucose and lipid homeostasis.

Another functionally conserved energy homeostatic pathway is the AMPK pathway. This pathway responds to altered energy states caused by exercise, low glucose, hypoxia, or mitochondrial inhibition (Carling, Trends Biochem. Sci. 29:18-24, 2004; Hardie, Curr. Opin. Cell Biol. 17:167-173, 2005; Kahn et al., Cell Metab. 1: 15-25, 2005). The increased AMP levels bind to the AMPK regulatory gamma subunit and prime the AMPKa kinase subunit for an activating phosphorylation mediated by the Peutz-Jegher tumor suppressor gene, LKB and possibly other AMPKKs (Shaw et al., Cancer Cell 6:91-99, 2004; Shaw et al., Proc. Natl. Acad. Sci. USA 101:3329-3335, 2004; Woods et al., Current Biol. 13:2004-2008, 2003). Activation of the energy-sensing AMPK pathway by an activated AMPK as well as metformin or AICAR treatment results in decreased lipogenesis and gluconeogenesis via both central and peripheral effects (Carling, Trends Biochem. Sci. 29:18-24, 2004; Hardie, Curr. Opin. Cell Biol. 17:167-173, 2005; Kahn et al., Cell Metab. 1:15-25, 2005). Also, activation of the AMPK within pancreatic beta cells leads to decreased insulin production (Leclerc and Rutter, Diabetes 53 (Suppl. 3):S67-74, 2004; Richards et al., J. Endocrinol. 187:225-235, 2005). These effects may be mediated by targets including glycogen synthase, hormone-sensitive lipase, acetylCoA carboxylase-2, and/or HMGCoA reductase (Carling, Trends Biochem. Sci. 29:18-24, 2004; Hardie, Curr. Opin. Cell Biol. 17:167-173, 2005; Kahn et al., Cell Metab. 1:15-25, 2005), although the different relationships of these and other proteins to the AMPK-mediated low energy response is not well known.

The Tuberous Sclerosis Complex (TSC1-2)/Target of Rapamycin (TOR) pathway has been shown to respond to changes in growth factors (like insulin/IGFs), amino acid levels, energy charge, lipid status, mitochondrial metabolites, and oxygen tension by adjusting cell growth (Abraham, Cell 111:9-12, 2002; Fingar and Blenis, Oncogene 23:3151-3171, 2004; Jacinto and Hall, Nature Rev. Molec. Cell Biol. 4:117-126, 2003; Kim and Sabatini, Curr. Topics Microbiol. Immunol. 279:259-270, 2004; Kozma and Thomas, Bioessays 24:65-71, 2002; Li et al., Trends Biochem. Sci. 29:32-38, 2004; Long et al., Curr. Top. Microbiol. Immunol. 279:115-138, 2004; Oldham and Hafen, Trends Cell. Biol. 13:79-85, 2003). In addition to its well-defined role in controlling cell growth, the TSC₁₋₂/TOR pathway may also potentially be a critical regulator of glucose and lipid homeostasis as TSC1-2/TOR functionally interacts with both the insulin/IGF and AMPK pathways (Wullschleger et al., Cell 124:471-484, 2006). As alterations of the insulin/IGF and AMPK pathways can lead to dramatically different metabolic effects, the direct role and function of TOR is unknown in this context.

There may be many levels where TSC₁₋₂/TOR signaling may positively and negatively regulate insulin signaling, yet how TSC₁₋₂/TOR signaling regulates glucose homeostasis and pancreatic beta cell function remains unclear, given the complexity of the possible levels of functional interactions with the insulin/IGF pathway. For example, a role for TOR signaling in glucose and lipid homeostasis in mammalian systems is supported by the S6K1 knock-out mice. These mice are hyperglycemic caused by diminished insulin secretion due to reduced pancreatic beta cell mass (Pende et al., Nature 408:994-997, 2000; Um et al., Nature 431:200-205, 2004). This result is in keeping with studies that showed that rapamycin treatment leads to decreased levels of translation, growth, and survival in pancreatic beta cells (Bell et al., Diabetes 52:2731-2739, 2003; Kwon et al., Diabetes 53 (Suppl. 3):S225-232, 2004; McDaniel et al., Diabetes 51:2877-2885, 2002; Xu et al., Diabetes 50:353-360, 2001; Xu et al., J. Biol. Chem. 273:4485-4491, 1998). However, the mS6K1 mutant mice show enhanced glucose uptake upon exogenous insulin addition due to insulin hypersensitivity in peripheral tissues, due to adipocytes that have increased fatty acid beta-oxidation (Um et al., Nature 431:200-205, 2004). Thus, TOR signaling can modulate insulin sensitivity at the level of IRS via Ser37 and Ser636/639 phosphorylation and altering IRS protein levels (Berg et al., Biochem. Biophys. Res. Comm. 293:1021-1027, 2002; Carlson et al., Biochem. Biophys. Res. Comm. 316:533-539, 2004; Haruta et al., Mol. Endocrinol. 14:783-794, 2000; Jaeschke et al., J. Cell Biol. 159:217-224, 2002; Khamzina et al., Endocrinology 146:1473-1481, 2005; Tremblay et al., Endocrinol. 146:1328-1337, 2005a; Tremblay et al., Diabetes 54:2674-2684, 2005b; Tzatsos and Kandror, Molec. Cell. Biol. 26:63-74, 2006; Ueno et al., Diabetologia 48:506-518, 2005; Um et al., Nature 431:200-205, 2004).

There is also data that suggests that the IRS Ser-302 site is positive and loss of phosphorylation by an unknown kinase may decrease insulin signaling to TOR and S6K (Giraud et al., J. Biol. Chem. 279:3447-3454, 2004). Thus, serine/threonine phosphorylation of the IRS proteins may be mediating both positive and negative downstream signals for energy homeostasis. In addition, Akt/PKB may also be negatively regulated directly by the nutrient-sensitive TOR pathway. Although the insulin/IGF pathway can signal to the TSC₁₋₂/TOR pathway, recent evidence suggests that TOR may alter Akt/PKB function because Akt/PKB activation depends on TORC2 complex-specific TOR Ser473 phosphorylation of Akt/PKB (Sarbassov et al., Science 307:1098-1101, 2005). Furthermore, increased AMPK activity can phosphorylate TSC2, which leads to decreased TOR activity, while loss of AMPK activity causes an increase in TOR activity (Bolster et al., J. Biol. Chem. 277:23977-23980, 2002; Dubbelhuis and Meijer, FEBS Lett. 521:39-42, 2002; Inoki et al., Cell 115:577-590, 2003; Kimura et al., Genes to Cells 8:65-79, 2003; Shaw et al., Science 310:1642-1646, 2005). Also, activation of AMPK leads to IRS Ser-789 phosphorylation and enhancement of insulin signaling (Jakobsen et al., J. Biol. Chem. 276:46912-46916, 2001). Thus, the contribution of the nutrient-sensing TSC₁₋₂/TOR pathway to the function of insulin-sending and insulin-receiving tissues is significant and likely complex. Clearly, there is a great need to understand the regulation of TSC₁₋₂/TOR signaling as it relates to the maintenance of energy homeostasis because dysregulation of TSC1-2/TOR signaling may contribute to the pathological progression of metabolic syndrome and diabetes.

Although TOR occupies a central node that governs catabolic or anabolic responses to different nutritional and energy states, the resultant metabolic effects of altering TOR function in a metazoan are incompletely and poorly understood. Many studies have made it clear that complete loss of TOR function is required for growth, yet these studies have not addressed the outcomes of reducing TOR function on energy metabolism, senescent responses, or functional interaction with the insulin pathway.

The role of TOR signaling in growth and metabolism is reviewed in Wullschleger et al., Cell 124:471-484, 2006.

U.S. Pat. No. 5,321,009 (Baeder et al.) discusses the treatment and prevention of insulin-dependent diabetes mellitus by administering rapamycin. U.S. Pat. No. 5,496,831 (Alexander-Bridges et al.) discusses the use of rapamycin to treat obesity and other complications caused by hyperinsulinemia. PCT Patent Application WO 2006/020755 (Cantley et al.) discusses the treatment of disorders characterized by reduced insulin responsiveness by administering an agent that decreases the activity of mTOR polypeptide, the TOR inhibitor rapamycin. Rapamycin can impair pancreatic beta cell function because it causes decreased growth and survival (Bell et al., Diabetes 52:2731-2739, 2003; McDaniel et al., Diabetes 51:2877-2885, 2002; Xu et al., Diabetes 50:353-360, 2001; Xu et al., J. Biol. Chem. 273:4485-4491, 1998). Thus, rapamycin treatment can lead to elevated glucose and lipid levels, possibly as a result of systemic insulin loss (Böhni et al., Cell 97:865-875, 1999; Burks et al., Nature 407:377-382, 2000; Kenyon et al., Nature 366:461-464, 1993; Kimura et al., Science 277:942-946, 1997). Additionally, although rapamycin affects TORC1 directly, TORC2 may be altered indirectly via TOR depletion (Sarbassov et al., Science 307:1098-1101, 2005). Thus, it is not currently clear how rapamycin is affecting TOR function.

### SUMMARY OF THE INVENTION

We have found that reducing the function of TOR results in decreased lipid stores and glucose levels (concomitant with increased production of ketone bodies in Drosophila). Furthermore, this reduction of TOR activity is able to block insulin resistance and metabolic syndrome phenotypes caused by expression of a constitutively activated version of the insulin responsive transcription factor, FOXO. These TOR-mediated responses are also linked with protection against age-dependent functional heart decline as well as increased longevity without changes in resistance to starvation and oxidative stresses. This profile is consistent with a unique TOR-mediated strategy to channel energy stores for the maintenance of organ and organismal function. Thus, this TOR response may represent an ancient “systems biological” response to regulate metabolism and senescence that has important evolutionary, physiological, and clinical implications. Based on these findings, compositions and methods are provided for modulating TOR activity and thereby treating metabolic syndrome and insulin resistance and related conditions. In addition, methods are provided for identifying substances that modulate TOR activity.

According to one aspect of the invention, compositions are provided that comprise an amount of a TOR inhibitor that is effective for treating one or more of the following: metabolic syndrome, insulin resistance, diabetes, obesity, cardiovascular disease, aging, and organ senescence.

According to another aspect of the invention, compositions are provided that comprise an amount of a TOR inhibitor that is effective for treating a condition in a patient selected from the group consisting of metabolic syndrome, insulin resistance, diabetes, obesity, cardiovascular disease, aging, and organ senescence, wherein the TOR inhibitor is effective in a rescue assay. Such TOR inhibitors include, for example, those that reduce glucose and/or lipid levels.

According to another aspect of the invention, compositions are provided that comprise an amount of a TOR inhibitor that is effective for treating a condition in a patient selected from the group consisting of metabolic syndrome, insulin resistance, diabetes, obesity, cardiovascular disease, aging, and organ senescence, wherein the TOR inhibitor reduces glucose levels in the patient. Such TOR inhibitors include, for example, those that also reduce lipid levels in the patient.

According to another aspect of the invention, compositions are provided that comprise an amount of a TOR inhibitor that is effective for treating a condition in a patient selected from the group consisting of metabolic syndrome, insulin resistance, diabetes, obesity, cardiovascular disease, aging, and organ senescence, wherein the TOR inhibitor reduces lipid levels in the patient.

According to another aspect of the invention, compositions are provided that comprise an amount of a TOR inhibitor that is effective for treating a condition in a patient selected from the group consisting of metabolic syndrome, insulin resistance, diabetes, obesity, cardiovascular disease, aging, and organ senescence, wherein the TOR inhibitor reduces lipid and glucose levels in the patient.

According to another aspect of the invention, compositions are provided that comprise an amount of a TOR inhibitor that is effective for treating a condition in a patient characterized by abnormal FOXO activity. Such TOR inhibitors include, for example, those that reduce glucose and/or lipid levels in the patient. In addition, such TOR inhibitors include those that are demonstrated to be effective in a rescue assay, as defined below.

According to another aspect of the invention, compositions are provided that comprise an amount of a TOR inhibitor that is effective in reducing glucose and lipid levels in a patient. In addition, such TOR inhibitors include those that are demonstrated to be effective in a rescue assay, as defined below.

According to another aspect of the invention, pharmaceutical compositions are provided that comprise any of the compositions mentioned above and a pharmaceutically acceptable carrier. Such pharmaceutical compositions may further comprise other suitable substances, including, for example, other active ingredients that are used for treating a particular condition.

According to another aspect of the invention, methods are provided for treating various conditions in a patient comprising administering to the patient an effective amount of a TOR inhibitor, such conditions including, but not limited to: metabolic syndrome, insulin resistance, diabetes, obesity, cardiovascular disease, aging, and organ senescence. In such methods the TOR inhibitor may have, for example, one or more of the following characteristics: effectiveness in a rescue assay; reduces glucose levels in the patient; and reduces lipid levels in the patient.

According to another aspect of the invention, methods are provided for treating a condition in a patient characterized by abnormal FOXO activity comprising administering to the patient a composition comprising an effective amount of a TOR inhibitor. Such conditions include, but are not limited to: metabolic syndrome, insulin resistance, diabetes, obesity, cardiovascular disease, aging, and organ senescence. In such methods, the TOR inhibitor may have, for example, one or more of the following characteristics: effectiveness in a rescue assay; reduces glucose levels in the patient; and reduces lipid levels in the patient.

According to another aspect of the invention, methods are provided for reducing glucose and lipid levels in a patient comprising administering to the patient a composition comprising an effective amount of a TOR inhibitor. In such methods, the TOR inhibitor may, for example, be effective in a rescue assay.

According to another aspect of the invention, methods are provided for identifying substances that are effective in treating a condition selected from the group consisting of metabolic disorder, insulin resistance, diabetes, obesity, cardiovascular disease, aging, and organ senescence. Such methods comprise (a) determining whether the substance is a TOR inhibitor, and (b) determining whether the substance is effective in treating the condition. The step of determining whether the substance is a TOR inhibitor may, for example, comprise, performing a rescue assay: (a) providing a cell (including, but not limited to, a Drosophila embryo, larva or adult), that has a mutation that confers a detectable phenotype in the presence of a TOR inhibitor (including, but not limited to a mutation is in one or more genes selected from the group consisting of TSC2, TSC2, AMPK, and LKB; a mutation that causes activation or overexpression of FOXO; and/or a mutation that causes overexpression of one or more proteins selected from the group consisting of Rheb, TOR or S6K); (b) contacting the cell with a composition comprising the substance; and (c) determining whether the cell displays the phenotype upon when contacted with the composition. Such methods may further comprise, for example, performing suitable assays to determine, for example, whether the substance reduces lipid and/or glucose levels.

According to another aspect of the invention, methods are provided for identifying a substance that is effective in treating a condition in a patient that is characterized by abnormal FOXO activity, the method comprising: (a) determining whether the substance is a TOR inhibitor; and (b) determining whether the substance is effective in treating the condition in the patient. Such conditions include, but are not limited to, the following: metabolic disorder, insulin resistance, diabetes, aging, and organ senescence.

According to another aspect of the invention, methods are provided for identifying a substance that is effective in reducing blood glucose levels in a patient, such methods comprising: (a) determining whether the substance is a TOR inhibitor; and (b) determining whether the substance is effective in reducing blood glucose levels in the patient. Such methods may further comprise, for example, performing suitable assays to determine, for example, whether the substance reduces lipid levels.

According to another aspect of the invention, methods are provided for identifying a substance that is effective in reducing lipid levels in a patient, such methods comprising: (a) determining whether the substance is a TOR inhibitor; and (b) determining whether the substance is effective in reducing lipid levels in the patient.

The foregoing and other aspects of the invention will become more apparent from the following detailed description, accompanying drawings, and the claims.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below.

### BRIEF DESCRIPTION OF THE FIGURES

FIG. 1 shows a sequence alignment of the dTOR⁷ allele (SEQ ID NO: 1) with the following TOR orthologs: human TOR (SEQ ID NO:2), yeast TOR1 (SEQ ID NO:3); yeast TOR2 (SEQ ID NO:4); Arabidopsis TOR (SEQ ID NO:5), and C. elegans TOR (SEQ ID NO:6). The mutated residue is D2116G and is completely conserved in TOR orthologs. This residue lies in the N-terminal region of the kinase domain and shows reduced kinase activity.

FIG. 2 shows a model of functional interaction of TOR signaling with insulin/IGF pathway. The numbered arrows indicate potential levels of functional interaction and are neither meant to represent whether the interaction is positive or negative nor any hierarchal dominance.

### DETAILED DESCRIPTION OF THE INVENTION

We have discovered that certain TOR inhibitors are useful in treating metabolic disorder, insulin resistance, diabetes, obesity, cardiovascular disease, and effects of aging and organ senescence. This result is surprising in view of the effects of the TOR inhibitor rapamycin and its derivatives, which can impair pancreatic beta cell function, because treatment with rapamycin causes decreased growth and survival and lead to elevated glucose and lipid levels, possibly as a result of systemic insulin loss.

As used herein, “TOR inhibitor” refers to any substance that inhibits TOR activity or function and any derivatives and pharmaceutically acceptable salts thereof. Rapamycin and rapamycin analogues are excluded from the TOR inhibitors according to the present invention: they impair pancreatic beta cell function and elevate glucose and lipid levels, which make them undesirable for the treatment of insulin resistance and metabolic syndrome phenotypes, for example. The mechanism of action of the TOR inhibitors of the invention is different from that of rapamycin. Functionally, TOR inhibitors according to the present invention are effective in treating conditions characterized by abnormal FOXO activity, including, but not limited to, metabolic disorder, insulin resistance, diabetes, obesity, cardiovascular disease, and various effects of aging and organ senescence. A rescue assay as described herein is useful for screening for such TOR inhibitors.

As used herein, “agent” refers to any substance that has a desired biological activity. For example, a “TOR inhibitory agent” or simply “TOR inhibitor” has detectable biological activity in inhibiting the function of TOR. In addition, TOR inhibitory agents have detectable biological activity, for example, in reducing insulin resistance, metabolic syndrome, diabetes, obesity, cardiovascular disease, aging and organ senescence, and symptoms thereof, in a host.

As used herein, “effective amount” refers to an amount of a composition that causes a detectable difference in an observable biological effect, including, but not limited to, a statistically significant difference in such an effect. The detectable difference may result from a single substance in the composition, from a combination of substances in the composition, or from the combined effects of administration of more than one composition. For example, an “effective amount” of a composition comprising a TOR inhibitor refers to an amount of the composition that detectably decreases TOR activity or function as measured directly in a suitable in vitro or in vivo assay. An effective amount of a TOR inhibitor may also refer to an amount of the composition that detectably measures or otherwise indicates in an indirect manner a decrease in TOR activity or function, e.g., a detectable biological effect in a cell or tissue, or systemically in an organism, including, but not limited to, a reduction in the magnitude or severity of metabolic syndrome, insulin resistance, diabetes, obesity, cardiovascular disease, aging and organ senescence, and related conditions, or one or more symptoms associated with the foregoing.

Compositions that comprise a TOR inhibitor according to the invention increase insulin levels; reduce glucose levels (e.g. blood glucose levels); reduce lipid levels (e.g., blood triglyceride or low-density lipoprotein [LDL] levels); and increase activity of a lipase and/or decrease lipid production by decreasing the activity of lipid-producing enzymes, such as FAS.

The term “metabolic syndrome” (also known as syndrome X or the dysmetabolic syndrome) designates a cluster of metabolic risk factors leading to cardiovascular disease that come together in a single individual. The main features of metabolic syndrome include insulin resistance, hypertension (high blood pressure), cholesterol abnormalities, and an increased risk for clotting. Patients are most often overweight or obese. Metabolic syndrome is associated with fat accumulation in the liver (fatty liver), resulting in inflammation and the potential for cirrhosis. The kidneys can also be affected, as there is an association with microalbuminuria, the leaking of protein into the urine, a subtle but clear indication of kidney damage. Other conditions associated with metabolic syndrome include obstructive sleep apnea, polycystic ovary syndrome, increased risk of dementia with aging, and cognitive decline in the elderly.

Besides changes in diet and exercise, patients suffering from metabolic syndrome are treated with drugs to control cholesterol levels, lipids, and high blood pressure. For example, a class of blood pressure drugs called ACE inhibitors has been found to also reduce the levels of insulin resistance and actually deter the development of type 2 diabetes. Drugs used to treat high blood sugar and insulin resistance may have beneficial effects on blood pressure and cholesterol profiles. A class of drugs called thiazolidinediones (pioglitazone [Actos] and rosiglitazone [Avandia] also reduce the thickness of the walls of the carotid arteries. Metformin (Glucophage), usually used to treat type 2 diabetes, also helps prevent the onset of diabetes in people with metabolic syndrome. A treatment regimen for a patient suffering from metabolic syndrome may combine administration of a composition comprising a TOR inhibitor according to the invention and administration of any other drug used to treat patients suffering form metabolic syndrome. For example, one may administer a single composition combining a TOR inhibitor and one or more of such other drugs. Alternatively, one may administer a composition comprising a TOR inhibitor together with or before or after administration of a different composition or compositions comprising one or more of such other drugs.

The term “insulin resistance” refers to the diminished ability of cells to respond to the action of insulin in promoting the transport of the sugar glucose, from blood into muscles and other tissues. Conditions associated with insulin resistance include, but are not limited to: type II diabetes; fatty liver; arteriosclerosis (or atherosclerosis) and such cardiovascular diseases as coronary artery disease, peripheral vascular disease, and strokes; skin lesions, including increased skin tags and acanthosis nigricans; reproductive abnormalities in women, including difficulty with ovulation and conception (infertility), irregular menses, or a cessation of menses; polycystic ovary disease; hyperandrogens; and growth abnormalities.

A combination of a TOR inhibitor and another active ingredient in a given composition or treatment may be a synergistic combination. The term “synergy,” as described for example by Chou and Talalay, Adv. Enzyme Regul. 22:27-55 (1984), occurs when the effect of the compounds when administered in combination is greater than the additive effect of the compounds when administered alone as a single agent. In general, a synergistic effect is most clearly demonstrated at suboptimal concentrations of the compounds. Synergy can be in terms of lower cytotoxicity, increased activity, or some other beneficial effect of the combination compared with the individual components. Such other active ingredients include, but are not limited to, any known composition that is used for treating a particular condition.

As used herein, to “treat” includes (i) preventing a pathologic condition from occurring (e.g. prophylaxis); (ii) inhibiting the pathologic condition or arresting its development; and (iii) relieving the pathologic condition; and/or preventing or reducing the severity one or more symptoms associated with such a pathologic condition. As one example, with respect to aging, treatment would not prevent aging, but would prevent or reduce the severity of one or more symptoms of aging. A composition comprising a TOR inhibitor is considered effective for treating metabolic syndrome or insulin resistance if it produces a detectable improvement (e.g., a reduction in) any of the conditions associated with metabolic syndrome or insulin resistance.

As used herein, the term “patient” refers to organisms to be treated by the compositions and methods of the present invention. Such organisms include, but are not limited to, mammals, including, but not limited to, humans, monkeys, dogs, cats, horses, rats, mice, etc. Such organisms also include other organisms, and cells, tissues and organs of such organisms, that are useful in screening for TOR modulators, including TOR inhibitors, including, but not limited to, yeast, Drosophila, Arabidopsis thaliana, Caenorhabditis elegans, etc. In the context of the invention, the term “subject” generally refers to an individual who will receive or who has received treatment (e.g., administration of a composition comprising a TOR inhibitor).

As used herein, “pharmaceutically acceptable salts” refer to derivatives of a TOR inhibitor or other disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like.

The pharmaceutically acceptable salts of a TOR inhibitor or other compounds useful in the compositions and methods of the present invention can be synthesized from the parent compound, which contains a basic or acidic moiety, by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., p. 1418 (1985), which is hereby incorporated by reference.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication commensurate with a reasonable benefit/risk ratio.

The TOR inhibitors can be administered as the parent compound, a pro-drug of the parent compound, or an active metabolite of the parent compound.

“Pro-drugs” are intended to include any covalently bonded substances which release the active parent drug or other formulas or compounds of the present invention in vivo when such pro-drug is administered to a mammalian subject. Pro-drugs of a compound of the present invention are prepared by modifying functional groups present in the compound in such a way that the modifications are cleaved, either in routine manipulation in vivo, to the parent compound. Pro-drugs include compounds of the present invention wherein the carbonyl, carboxylic acid, hydroxy or amino group is bonded to any group that, when the pro-drug is administered to a mammalian subject, cleaves to form a free carbonyl, carboxylic acid, hydroxy or amino group. Examples of pro-drugs include, but are not limited to, acetate, formate and benzoate derivatives of alcohol and amine functional groups in the compounds of the present invention, and the like.

“Metabolite” refers to any substance resulting from biochemical processes by which living cells interact with the active parent drug or other formulas or compounds of the present invention in vivo, when such active parent drug or other formulas or compounds of the present are administered to a mammalian subject. Metabolites include products or intermediates from any metabolic pathway.

“Metabolic pathway” refers to a sequence of enzyme-mediated reactions that transform one compound to another and provide intermediates and energy for cellular functions. The metabolic pathway can be linear or cyclic.

Numerous modifications and variations of the present invention are possible in light of the above teachings. It is therefore to be understood that within the scope of the appended claims, the invention may be practiced otherwise than as specifically described herein.

### Inhibitors of TOR; Screening Methods

A number of inhibitors of TOR are known in the literature. At one time it was hypothesized that the TOR inhibitor rapamycin (sirolimus, Rapamune® [Wyeth Pharmaceuticals Inc]) and its derivatives (Easton and Houghton, Expert Opin. Ther. Targets 8(6):551-564, 2004; Webster et al., Cochrane Database Syst. Rev. 2006 (2), article no. CD004290), including, but not limited to CCl-779 (Temsirolimus), RAD001 (Everolimus) and AP23576 (Vignot et al., Ann. Oncology 16:525-537, 2005; Webster et al., Cochrane Database Syst. Rev. 2006 (2), article no. CD004290; Eisen et al., New England J. Med. 349:847-858, 2003) would be useful for treating diabetes and insulin resistance. However, rapamycin can impair pancreatic beta cell function because it causes decreased growth and survival (Bell et al., Diabetes 52:2731-2739, 2003; McDaniel et al., Diabetes 51:2877-2885, 2002; Xu et al., Diabetes 50:353-360, 2001; Xu et al., J. Biol. Chem. 273:4485-4491, 1998). Rapamycin treatment can lead to elevated glucose and lipid levels, possibly as a result of systemic insulin loss (Böhni et al., Cell 97:865-875, 1999; Burks et al., Nature 407:377-382, 2000; Kenyon et al., Nature 366:461-464, 1993; Kimura et al., Science 277:942-946, 1997). Rapamycin and rapamycin derivatives are used clinically for immunosuppression, such as in preventing organ rejection in transplant patients, for example, which would generally be considered an undesirable side effect with respect to treatment of metabolic disorder, insulin resistance, diabetes, obesity, cardiovascular disease, and symptoms of aging and organ senescence.

As a result, methods are provided for screening for other substances that are effective inhibitors of TOR and have an acceptable safety profile. In addition to the methods taught herein, methods for screening for TOR inhibitors are known in the art, including, for example, the methods discussed in U.S. Patent Application 20040191836 (Abraham) and PCT Patent Application WO 2006/020755 (Cantley et al.). See also Knight et al., Cell 125:1-15 (2006), which describes synthesis and assays for a number of inhibitors of phosphatidylinositol 3-kinases (PI3-Ks), including inhibitors of mTOR.

“Rescue assay” refers to a biological assay that relies on the ability of TOR inhibitors according to the invention to reduce or reverse the effects of certain mutations. Rescue assays employ suitable, scorable mutant phenotypes (lethality, growth, fluorescence, metabolic changes, stress alterations, etc.) caused by a mutation of TSC1, TSC2, AMPK or LKB; a mutation that activates FOXO; or a mutation that results in overexpression of Rheb, TOR or S6K. Such mutations are known in Drosophila and other organisms. Test compounds are added to the medium of cells having the relevant mutant phenotype (here, “cell” includes tissues, embryos, larvae, etc. that are made up of such mutant cells) and the degree of rescue or reversion of the mutant phenotype in response to the TOR inhibitor is scored by appropriate means, including but not limited to, microscopic, photographic, cell biological, enzymatic, or other methods. For example, based on our observation that complete loss of dTOR function in Drosophila results in early larval lethality, and the ability of TOR inhibitors, we have developed a high-throughput assay for TOR inhibitors by placing mutant Drosophila embryos or larvae that are arrested in development in S2 or another appropriate cell medium in a multi-well plate and observing the ability of a TOR inhibitor to rescue the embryos or larvae, that is, to permit them to continue to develop further. Similar assays may be performed using mammalian or other cells or tissues that have similar genetic lesions.

It is expected that TOR inhibitors that rescue Drosophila embryos in our high-throughput assay will also function similarly in mammals, including humans, given the many genetic, biochemical and phenotypic similarities between Drosophila and mammalian systems with respect to insulin signaling and the regulation of energy homeostasis.

Pharmaceutical Compositions and Methods.

The compositions of the invention can be formulated as pharmaceutical compositions and administered to a mammalian host, such as a human patient, in a variety of forms adapted to the chosen route of administration, i.e., orally or parenterally, by intravenous, intramuscular, topical or subcutaneous routes.

Such compositions may be systemically administered in vivo by a variety of routes. For example, they may be administered orally, in combination with a pharmaceutically acceptable excipients such as an inert diluent or an assimilable edible carrier. They may be enclosed in hard or soft shell gelatin capsules, may be compressed into tablets, or may be incorporated directly with the food of the patient's diet. For oral administration, the active ingredient or ingredients may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active ingredient in such useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The compositions may also be administered intravenously or intraperitoneally by infusion or injection. Solutions of a TOR inhibitor, its salts and other active ingredients can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating a TOR inhibitor or other active ingredients in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, a TOR inhibitor and other active ingredients may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Useful dosages of a TOR inhibitor or other active ingredients can be determined by comparing their in vitro activity and in vivo activity in animal models. Methods for the extrapolation of effective dosages in mice, and other animals, to humans are known to the art; for example, see U.S. Pat. No. 4,938,949.

Generally, the concentration of a TOR inhibitor or other active ingredients of the invention in a liquid composition will be from about 0.1-25 wt-%, preferably from about 0.5-10 wt-%. The concentration in a semi-solid or solid composition such as a gel or a powder will be about 0.1-5 wt-%, preferably about 0.5-2.5 wt-%.

The amount of the compound, or an active salt or derivative thereof, required for use alone or with other agents will vary not only with the particular salt selected but also with the route of administration, the nature of the condition being treated and the age and condition of the patient and will be ultimately at the discretion of the attendant physician or clinician.

In general, however, a suitable dose may be in the range of from about 0.5 to about 100 mg/kg, e.g., from about 1 to about 75 mg/kg of body weight per day, or 1.5 to about 50 mg per kilogram body weight of the recipient per day, or about 2 to about 30 mg/kg/day, or about 2.5 to about 15 mg/kg/day.

The compound may be conveniently administered in unit dosage form; for example, containing 5 to 1000 mg, conveniently 10 to 750 mg, most conveniently, 50 to 500 mg of active ingredient per unit dosage form.

The active ingredient may be administered to achieve peak plasma concentrations of the active compound of from about 0.5 to about 75 μM, preferably, about 1 to 50 μM, most preferably, about 2 to about 30 μM. This may be achieved, for example, by the intravenous injection of a 0.05 to 5% solution of the active ingredient, optionally in saline, or orally administered as a bolus containing about 1-100 mg of the active ingredient. Desirable blood levels may be maintained by continuous infusion to provide about 0.01-5.0 mg/kg/hr or by intermittent infusions containing about 0.4-15 mg/kg of the active ingredient(s).

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

Pharmaceutical compositions according to the invention may comprise one or more than one TOR inhibitors. Pharmaceutical compositions comprising a TOR inhibitor may also include other active ingredients including, but not limited to, insulin and Metformin (Glucophage) and analogs, for example.

The present invention will be further described by the following nonlimiting examples.

### EXAMPLE 1

We have examined in detail the function of Drosophila TOR (dTOR) in terms of energy homeostasis and senescent responses. We show that reduction of dTOR function results in decreased glucose and lipid levels with concomitant increase of the Drosophila insulin-like peptides (DILPs) from the insulin-producing cells. We also show that a reduction of dTOR function can block activated dFOXO-mediated insulin resistance and metabolic syndrome phenotypes. Taken together, these data indicate that dTOR function is required for the maintenance of energy homeostasis and organismal senescence. Reduction of TOR function has clinical utility for treating metabolic syndrome and insulin resistance.

### Materials and Methods

Fly Genetics. Standard genetic analyses were performed as described (Oldham et al., Genes Dev. 14:2689-2694, 2000).

Immunohistochemistry, Westerns and RT-PCR Analyses. We used the DILP2 antibody to measure DILP2 protein levels by performing immunohistochemistry as described (Rulifson et al., Science 296:1118-1120, 2002) and wt-d₄EBP, phospho-4EBP, wt-dS6K, and phospho-dS6K antibodies to perform Western blotting as described (Miron et al., Nature Cell Biol. 3:596-601, 2001; Miron et al., Molec. Cell. Biol. 23:9117-9126, 2003; Oldham et al., Genes Dev. 14:2689-2694, 2000). We isolated total RNA (Trizol kit, Invitrogen, according to manufacturer's instructions) and constructed cDNA libraries (QuantiTect Reverse Transcription kit, Qiagen, according to manufacturer's instructions) from the yw control, dFOXO-TM, the dTOR^{7/P} mutant, and combinations. We then performed semi-quantitative RT-PCR using primers that spanned the DILP2, dFAS, or brummer intron and used actin5C as an internal loading control. All PCR DNA amplifications were run using the following conditions; only the number of cycles and annealing temperatures are different for each primer set: 94° C. for three minutes; number of cycles variable, 94° C. for 20 sec; variable annealing temperature, 72° C. for 20 sec; 72° C. 5 minutes. For analyzing mRNA levels of the lipase gene, we used the following primers: forward primer, 5′-ACT ACA CGA CGT AGC C-3′ (SEQ ID NO: 7); reverse primer, 5′-CAA CCT CGG GAT CAC T-3′ (SEQ ID NO: 8). The expected product size was 231 bp. The PCR conditions used were: denaturation at 94° C. for three minutes, annealing at 56° C., and extension at 72° C. for 20 sec for 30 cycles plus a five minute final extension For analyzing mRNA levels of the DILP2 gene, we used the following primers: forward primer, 5′-CTG GGA GCT ATC TTG GGG GTG-3′ (SEQ ID NO: 9); reverse primer, 5′-CGC GTC GAC CAG GTC AGT TG-3′ (SEQ ID NO: 10). The expected product size was 193 bp. The PCR conditions used were: denaturation at 94° C. for three minutes, annealing at 55° C., and extension at 72° C. for 20 sec for 26 cycles plus a five minute final extension. For analyzing mRNA levels of the dFAS gene, we used the following primers: forward primer, 5′-GAC GAA TGT GAC CTT GG-3′ (SEQ ID NO: 11) and reverse primer, 5′-ACC CGA GTA TTG GGT GA-3′ (SEQ ID NO: 12). The expected product size was 281 bp. The PCR conditions used were: denaturation at 94° C. for three minutes, annealing at 50° C., and extension at 72° C. for 20 sec for 35 cycles plus a five minute final extension. For analyzing mRNA levels of the actin 5C gene, we used the following primers: forward primer, 5′-TAC CCC ATT GAG CAC GGT AT-3′ (SEQ ID NO: 13); reverse primer, 5′-GGT CAT CTT CTC ACG GTT GG-3′ (SEQ ID NO: 14). The expected product size was 200 bp. The PCR conditions used were: denaturation at 94° C. for three minutes, annealing at 55° C., and extension at 72° C. for 20 sec for 27 cycles plus a five minute final extension.

Metabolic Assay for Lipid and Glucose: We isolated the fat body from third instar larvae, fixed them with formaldehyde, and stained them with 0.5 mg/ml (final) Nile Red. The fat body were washed with 1×PBS and then mounted in Vectashield for fluorescence microscopy (Zeiss, Axiophot). Triglyceride and glucose levels were detected using a glucose oxidase assay (Pointe) and lipid assay (Thermo) as described (Rulifson et al., Science 296:1118-1120, 2002; Teleman et al., Genes Dev. 19:1844-1848, 2005) with exception that we extracted the lipids with a 1:1 mixture of chloroform/methanol (vol:vol).

Metabolic Assay for Ketone Bodies: The beta-hydroxybutyrate (bHB) ketone body was measured as follows. We used 20 mg of tissue weighed on a balance cooled with cups of liquid N₂ around the balance pan. The tissue was weighed into a 1.5 or 2.0 ml screw cap Eppendorf centrifuge tube containing glass beads (1 mm diameter) that was cooled in liquid nitrogen. To the tissue, four volumes volumes of ice-cold 0.55 M perchloric acid were added and homogenized immediately for 30 sec using a Minibeadbeater (Biospec Products). The samples were then neutralized by adding 3M KHCO₃. After neutralizing, the tubes were placed in ice for 30-60 min and spun and the supernatant was transferred to a screw cap cent tube for analysis. For the fluorometric assay the wells contained: 50 mM 2-amino-2-methylpropanol (pH 9.9), 5 mM NAD, 2 mM EDTA, 0.01% BSA. Analyses were done using a TECAN Spectrafluor Plus microplate reader (Durham, N.C.) using a 340 nm excitation filter and a 465 nm emission filter. We used bHB dehydrogenase (Biocatalytic) and NADH production from bHB was measured.

Assay for Starvation: Survival of male flies was tested under water-only starvation conditions as described in (Oldham et al., Genes Dev. 14:2689-2694, 2000).

Assay for ROS resistance: Survival of male flies was tested during exposure to paraquat (Pq)-induced oxidative stress. Flies were exposed to 10 mM Pq in 1% sucrose and survival was assessed after 48 hours.

Assay for Lifespan: Lifespan assays were performed by taking 15-20 flies per tube and turning to a fresh tube every 2-3 days. The number of viable flies was scored at these intervals.

Assay for Heart Function: Cardiac failure rates in response to electrical pacing were measured according to Wessels et al. (Nature Genetics 36:1275-1281, 2004).

### Results

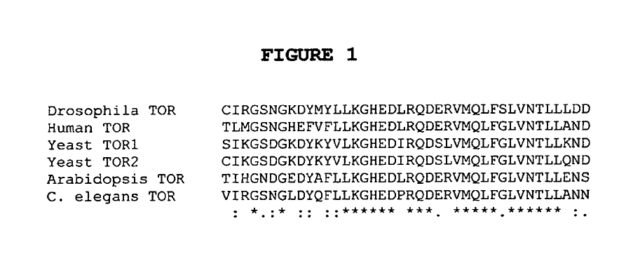

Complete loss of dTOR function results in early larval lethality (Oldham et al., Genes Dev. 14:2689-2694, 2000; Zhang et al., Genes Dev. 14:2712-2724, 2000). In order to analyze the effects of a reduction of dTOR function on processes like energy homeostasis and senescence, we utilized a new hypomorphic mutation in dTOR within the kinase domain (Oldham et al., Genes Dev. 14:2689-2694, 2000). FIG. 1 shows a sequence alignment of the dTOR⁷ allele with TOR orthologs from humans, yeast, Arabidopsis, and Caenorhabditis elegans. The dTOR^{2L7} lesion is an aspartic acid to glycine substitution (D2116G) in the N-terminal region of the kinase domain, and the dTOR^{2L15} allele is a glutamic acid to lysine mutation (E1568K) within the FAT domain. The two missense mutations occur in highly conserved stretches of amino acids that are in highly conserved domains across all species. There are slightly reduced phospho-dS6K (T398) and no wild-type d4EBP levels in the dTOR^{7/P} mutant flies compared to control as determined by Western blotting, although dFAS mRNA levels are not changed in the dTOR^{7/P} mutant flies.

Flies transheterozygous for this dTOR allele (dTOR^{2L7}) and a dTOR P-element insertion (dTOR^{1(2)k17004}, hereafter called dTOR^{7/P} mutant) are viable and 20% smaller compared to control. We first examined how dTOR effectors are altered in the dTOR^{7/P} mutant. Unexpectedly, we observed no wild-type d4EBP protein and only mildly decreased phospho-dS6K compared to the control. This effect may be due to translational or post-translational effects, as we observe d4EBP mRNA in the dTOR^{7/P} mutant.

Depending on the type of environmental perturbation, lipid levels may be increased for diapause (as seen in InR pathway mutants) or hibernation, whereas during times of energy reduction caused by dieting or exercising (which leads to AMPK activation), lipids are mobilized preferentially over protein stores for fuel because of their high energy content (Cahill and Veech, Trans. Amer. Clin. Climatological Assoc. 114:149-161, discussion 162-143, 2003; Saltiel and Kahn, Nature 414:799-806, 2001). As TOR is a central component in response to fluctuations in amino acids, growth factors, oxygen tension, and energy status (Fingar and Blenis, Oncogene 23:3151-3171, 2004; Hafen, Curr. Top. Microbiol. Immunol. 279:153-167, 2004; Kozma and Thomas, Bioessays 24:65-71, 2002; Li et al., Trends Biochem. Sci. 29:32-38, 2004; Long et al., Curr. Top. Microbiol. Immunol. 279:115-138, 2004; Wullschleger et al., Cell 124:471-484, 2006), we first determined how metabolic stores are altered by examining the lipid levels in the dTOR^{7/P} mutant. It has been shown that systemic loss of insulin signalling leads to increased lipid levels in worms, flies, and mammals (Böhni et al., Cell 97:865-875, 1999; Burks et al., Nature 407:377-382, 2000; Kenyon et al., Nature 366:461-464, 1993; Kimura et al., Science 277:942-946, 1997).

Surprisingly, reduction of dTOR function with the heteroallelic combination caused a decrease in lipid levels in the fat body. Drosophila TOR regulates lipid metabolism. Nile Red staining of control third instar larvae fatbody and of the dTOR^{7/P} mutant larvae fatbody revealed substantially decreased lipid levels in the fat body of the dTOR^{7/P} mutant compared to control. The remaining lipids were confined to small vesicles within the cytoplasm of the fatbody cells.

These decreased lipid levels in the dTOR^{7/P} mutant may be due to an increased utilization of lipids from pre-existing lipid stores and/or the prevention of lipid storage. This decrease in lipid levels correlated with an increase in the mRNA levels of a lipase (termed brummer in Drosophila) involved in regulating lipid levels in the fatbody (Grönke et al., Cell Metabolism 1:323-330, 2005), which suggests that reduction of dTOR function results in lipid breakdown due to increased lipase activity. We found that reduction of dTOR function resulted in increased lipase mRNA levels. Consistent with the elevated lipase mRNA levels, we also observed a 27% decrease in triglyceride levels in the dTOR^{7/P} mutant that depended on the lipase gene as a null mutant for brummer reversed the triglyceride decrease. We also saw no change in the levels of dFAS mRNA levels, which supports the idea that reducing dTOR function results in increased lipid breakdown. To further determine the basis of the lipid breakdown, we measured ketone bodies (beta-hydroxybutyrate), whose levels also serve as an indicator of lipid utilization. Reduction of dTOR function results in elevated ketone bodies (p=0.0028, unpaired, two-tailed t-test; this experiment was performed two times independently). The data show that there was a 155% increase in beta-hydroxybutyrate levels in the dTOR^{7/P} mutant flies, which is consistent with a conversion of lipids to ketone bodies. Thus, reduction of dTOR function causes the breakdown of lipid stores and conversion into ketone bodies.

Changes in lipid metabolism are linked with alterations in insulin signaling (Saltiel and Kahn, Nature 414:799-806, 2001; Wu et al., Molec. Cell 3:151-158, 1999). Drosophila contains seven Drosophila insulin-like peptides, which are structurally and functionally related to insulin and IGF (Brogiolo et al., Curr. Biol. 11:213-221, 2001). The bilateral set of seven medial neurosecretory insulin producing cells (IPCs) in the fly brain hemispheres express DILP2 (Ikeya et al., Curr. Biol. 12:1293, 2002; Rulifson et al., Science 296:1118-1120, 2002). Loss of the IPCs increase blood glucose levels and can be rescued by exogenous DILP2 expression, which shows that loss of insulin/IGF production and signaling is causally linked to altered glucose homeostasis in Drosophila (Broughton et al., Proc. Natl. Acad. Sci. USA 102:3105-3110, 2005; Ikeya et al., Curr. Biol. 12:1293, 2002; Rulifson et al., Science 296:1118-1120, 2002).

As DILP2 is a key regulator for glucose levels, we first determined whether DILP2 protein levels are changed in the neurosecretory IPCs in the dTOR^{7/P} mutant. We found that Drosophila TOR regulates DILP2 and glucose metabolism. DILP2 showed expression in the cytoplasm of the NSC cell body and into the axonal processes in the wild-type. The dTOR^{7/P} mutant had increased DILP2 staining in both the cell body and axons (as demonstrated by confocal Z-series taken at 60×). We saw that the dTOR^{7/P} mutant resulted in an increase in DILP2 protein levels in both the cell body and axonal projections of the IPCs.

To determine if the increased DILP2 protein levels due to reducing dTOR function also occurs at the mRNA level, we performed RT-PCR on the dTOR^{7/P} mutant and looked for changes in DILP2 mRNA levels. DILP2 mRNA levels are increased in the dTOR^{7/P} mutant flies. Remarkably, we saw that the dTOR^{7/P} mutant had increased DILP2 mRNA levels relative to the control. Thus, reducing dTOR function can increase the levels of DILP2. As altered DILP2 levels lead to changes in glucose homeostasis, we next examined the dTOR^{7/P} mutant for possible effects on glucose homeostasis. The dTOR⁷/p mutant glucose levels were significantly decreased (p=0.0086, unpaired, two-tailed t-test) compared to control. Remarkably, the dTOR^{7/P} mutant showed a 39% decrease in blood glucose levels. Thus, the dTOR^{7/P} mutant is strongly hypoglycemic and a reduction of dTOR function can lead to lowered glucose levels.

The insulin/IGF pathway is a major integrator of metabolic and stress signals that depend on the function of FOXO (Matsumoto and Accili, Cell Metabol. 1:215-216, 2005). Constitutively activated FOXO can increase the lifespan of worms and flies if expressed in certain spatiotemporal patterns (Giannakou et al., Science 305:361, 2004; Hwangbo et al., Nature 429:562-566, 2004; Libina et al., Cell 115:489-502, 2003; Wolkow et al., Science 290:147-150, 2000). However, constitutive FOXO activation in liver and pancreatic beta cells causes hyperglycemia in mammals (Nakae et al., Nature Genet. 32:245-253, 2002). Thus, activated FOXO represents the most distal step known for inducing insulin resistance and metabolic syndrome phenotypes.

Because decreased TOR signaling can enhance insulin signaling upstream of FOXO via reduced IRS serine/threonine phosphorylation, we used a constitutively active dFOXO (dFOXO-TM, in which the three Akt/PKB phosphorylation sites have been inactivated) to ask whether reducing dTOR function can act downstream of dFOXO function (Hwangbo et al., Nature 429:562-566, 2004; Junger et al., J. Biol. 2:20, 2003; Kramer et al., BMC Dev. Biol. 3:5, 2003; Puig et al., Genes Dev. 17:2006-2020, 2003). We first analyzed systemic dFOXO expression for changes in lipid and glucose levels. We saw that expression of activated dFOXO resulted in increased lipid levels as assessed by Nile Red staining and measurement of triglyceride levels. The arm-Gal4; UAS-dFOXO-TM line had increased lipid levels, while the arm-Gal4, dTOR^{7/P}; UAS-dFOXO-TM mutant combination had lipid levels similar to the dTOR^{7/P} single mutant (all lines were significantly different (p<0.05, unpaired, two-tailed t-test) relative to the control. These elevated lipid levels corresponded with increased dFAS mRNA levels in this background. We next asked whether the dTOR^{7/P} mutant is able to alter this activated dFOXO metabolic phenotype. We found that a reduction of dTOR function can block both the lipid and glucose increase mediated by constitutively activated dFOXO. We also saw that dFAS mRNA levels are decreased in the activated dFOXO-dTOR^{7/P} mutant background. We saw that dFOXO was still present in the nucleus, so the dTOR^{7/P} mutant is not altering the localization of dFOXO. As activated dFOXO expression in the head fatbody decreases DILP2 mRNA levels (Hwangbo et al., Nature 429:562-566, 2004), we next determined the effect of expressing activated dFOXO specifically in the IPCs. We saw that activated dFOXO-mediated insulin resistance within the IPCs led to low DILP2 mRNA levels and high glucose levels caused by constitutively activated dFOXO expression in the insulin-producing cells are blocked by the dTOR^{7/P} mutant. Thus, in addition to upstream effects on insulin pathway components, reducing dTOR function can block dFOXO-mediated relevant metabolic targets to reverse the increased glucose and lipid phenotypes.

We next analyzed whether reducing dTOR function can have beneficial effects on aging and organ senescence. As TOR integrates growth factors (like insulin/IGFs), amino acids levels, energy charge, lipid status, mitochondrial metabolites, and oxygen tension, TOR responds to many stimuli that can potentially affect both longevity and organ senescence (Houthoofd et al., J. Gerontol. Ser. A-Biol. Sci. & Med. Sci. 60:1125-1131, 2005; Kenyon, Cell 120:449-460, 2005; Partridge and Gems, Nat. Rev. Genet. 3:165-175, 2002; Sharp and Bartke, J. Gerontol. Ser. A-Biol. Sci. & Med. Sci. 60:293-300, 2005; Tatar et al., Science 299:1346-1351, 2003). Indeed, studies in Drosophila using over-expression of dTOR pathway inhibitors, suggest that TOR signalling may be contributing to aging (Kapahi et al., Current Biol. 14:885-890, 2004). Furthermore, direct inhibition of TOR in yeast and worm show that loss of TOR function results in increased lifespan along with increased stress resistance (Kaeberlein et al., Science 310:1193-1196, 2005; Powers et al., Genes Dev. 20:174-184, 2006; Vellai et al., Nature 426:620, 2003).

We asked whether the dTOR^{7/P} mutant could alter aging at the organ and organismal level, by first using an assay that measures the progressive age-dependent decline in heart function (Wessells et al., Nature Genet. 36:1275-1281, 2004). We found that dTOR regulates age-related changes in cardiac performance. The failure rate of wild-type flies (yw) changes significantly with age (age-by-genotype, chi squared 59.2, p<0.0001). The cardiac failure rate of the dTOR^{7/P} mutant exhibited a significantly decreased rate of change with age compared to yw (age-by-genotype, chi squared=7.49, p=0.0519). Thus we found that the dTOR^{7/P} mutant had a low heart failure rate that was observed in young flies as well as old flies. Remarkably, the heart failure rate of old dTOR^{7/P} mutant flies was similar to the heart failure rate of the young flies.

We also found that the dTOR^{7/P} mutant flies exhibited a significantly extended lifespan as compared to the yw background (chi squared=12.42, p=0.0004) and to the dTOR^{7/P} mutant flies which also carried a genomic rescue construct for dTOR (chi squared=10.56, p=0.0012). The dTOR⁷/p mutant flies with a dTOR rescue construct no longer showed a significant difference from the yw background (chi squared=0.15, p=0.7001). Thus, in keeping with the decrease in heart failure, the median lifespan of the dTOR^{7/P} mutant was increased by 20% under normal feeding conditions. Thus, the age-related protection of heart function correlates well with the increased lifespan in the dTOR^{7/P} mutant.

As increased longevity is frequently associated with increased stress resistance (Kenyon, Cell 120:449-460, 2005), we tested whether the dTOR^{7/P} mutant can resist environmental stresses. We first tested the ability of the dTOR^{7/P} mutant to withstand water-only starvation. Remarkably, a reduction in dTOR activity had no effect on starvation resistance. The dTOR^{7/P} mutant does not affect resistance to starvation conditions compared to the background yw genotype (genotype effect, chi squared=1.23, p=0.2676). Therefore, there is no starvation sensitivity, despite decreased lipid stores caused by a reduction of dTOR function.

We next tested dTOR^{7/P} for its ability to resist ROS production induced by paraquat. Remarkably, a reduction in dTOR activity had no effect on resistance to oxidative stress. The dTOR^{7/P} mutant had no significant effect on survival compared to the wild-type yw stock (unpaired, two-tailed t-test, p=0.477). Thus, reduction of dTOR function does not provide resistance against acute stresses. We also observed that the dTOR^{7/P} mutants are fertile, lack significant developmental delay, have wild-type levels of ATP, and retain flight ability. In contrast to the strong association between increased longevity and acute stress resistance, these results suggest that dTOR may preferentially regulate organ senescence and longevity versus acute stress responses.

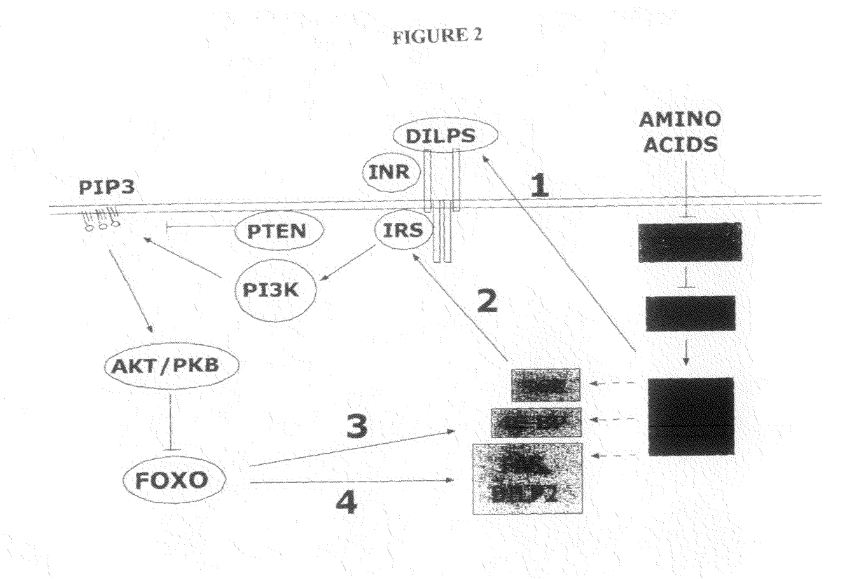

FIG. 2 shows a model of functional interaction of TOR signalling with insulin/IGF pathway. The numbered arrows indicate potential levels of functional interaction and are neither meant to represent whether the interaction is positive or negative nor any hierarchal dominance.

### Discussion

In contrast to the elevated lipid levels caused by reduction of systemic insulin signaling (Böhni et al., Cell 97:865-875, 1999; Burks et al., Nature 407:377-382, 2000, Kenyon et al., Nature 366:461-464, 1993; Kimura et al., Science 277:942-946, 1997), the dTOR⁷/p mutant does not show increased lipid levels. Instead, the dTOR^{7/P} mutant shows decreased lipid levels of the fat body that depend on the function of a lipase involved in lipid metabolism (Grönke et al., Cell Metabolism 1:323-330, 2005). We also observed elevated ketone bodies in the hypoglycemic dTOR^{7/P} mutant, which is indicative of the increased utilization of lipids. Studies in mammalian cardiac tissue have shown that ketone bodies provide their high energy electrons directly to complex I, the NADH dehydrogenase multi-enzyme complex, of the mitochondrial electron transport chain (Cahill and Veech, Trans. Amer. Clin. Climatological Assoc. 114:149-161, discussion 162-143, 2003). This bypass increases the ΔG of ATP formation due to a more reduced complex I and a more oxidized complex III, which increases the energy of the proton gradient (Cahill and Veech, Trans. Amer. Clin. Climatological Assoc. 114:149-161, discussion 162-143, 2003). Interestingly, many amino acids are utilized for energy production, either through the TCA cycle and/or ketone body formation. Thus, the altered lipid levels show that dTOR has a critical role in determining the fate of fats.

It has also been shown that dTOR and d4EBP in Drosophila are involved in lipid metabolism because the increased lipid levels caused by rapamycin treatment are blocked by a d4EBP mutant (Teleman et al., Genes Dev. 19:1844-1848, 2005). We see that the dTOR^{7/P} mutant behaves differently than rapamycin treatment, which suggests that rapamycin does not equal the dTOR^{7/P} mutant lipid-lowering phenotype. This difference is important. In support of this idea, it is known that rapamycin can impair pancreatic beta cell function because it causes decreased growth and survival (Bell et al., Diabetes 52:2731-2739, 2003; McDaniel et al., Diabetes 51:2877-2885, 2002; Xu et al., Diabetes 50:353-360, 2001; Xu et al., J. Biol. Chem. 273:4485-4491, 1998). Thus, rapamycin treatment can lead to elevated glucose and lipid levels, possibly as a result of systemic insulin loss (Böhni et al., Cell 97:865-875, 1999; Burks et al., Nature 407:377-382, 2000; Kenyon et al., Nature 366:461-464, 1993; Kimura et al., Science 277:942-946, 1997). Additionally, although rapamycin affects TORC1 directly, TORC2 may be altered indirectly via TOR depletion (Sarbassov et al., Science 307:1098-1101, 2005). Thus, it is not currently clear how rapamycin is affecting TOR function. Nevertheless, we see that a novel hypomorphic dTOR FAT domain allele in combination with the dTOR^{P} allele also shows low glucose and lipid levels, which suggests that reduction of dTOR activity represents a unique phenotypic class of dTOR metabolic effects versus an allele specific phenotype.

DILP2 levels are increased in the IPCs of the dTOR^{7/P} mutant, and the dTOR^{7/P} mutant has lowered glucose levels. Thus, reduction of dTOR function can lead to increased DILP2 levels and a reduction of glucose levels. Recent studies with the miRNA-278 mutant also showed elevated glucose and DILP levels in response to fatbody-mediated insulin resistance as shown by elevated 4EBP levels (Teleman and Cohen, Genes Dev. 20:417-422, 2006). We believe that the dTOR^{7/P} mutant represents an insulin-sensitized state because the dTOR^{7/P} mutant shows decreased levels of the insulin resistance marker d4EBP, the dTOR^{7/P} mutant shows decreased glucose levels, and, as discussed below, the dTOR^{7/P} mutant blocks activated dFOXO-mediated insulin resistance phenotypes. Thus, the dTOR mutant phenotype resembles a whole-animal “insulin-sensitized” state that can function below the level of constitutive dFOXO activity.

Reduction of TOR activity inhibits activated FOXO metabolic phenotypes. We see that overexpression of activated dFOXO in peripheral and IPC tissues results in elevated glucose and lipid levels. These effects require dTOR as reducing dTOR function is able to reverse these effects. Thus, our results show that the dTOR response is downstream of the dFOXO-mediated insulin resistance and metabolic syndrome phenotypes, which suggests that strategies to dampen, reduce, or block TOR signaling may be able to overcome insulin resistance (i.e., hyperglycemia and hypertriglyceridemia) below the level of increased FOXO activity in mammalian systems.

Although FOXO has greater than one hundred potential targets that might contribute to the metabolic phenotype (Lee et al., Science 300:644-647, 2003; McElwee et al., Aging Cell 2:111-121, 2003; Murphy et al., Nature 424:277-283, 2003), we have identified dFAS and DILP2 as candidate mediators of the dTOR effect on the dFOXO metabolic phenotypes. The effect on dFAS is interesting because it is upregulated by dFOXO overexpression and in an IRS/chico mutant and may be an important determinant of the lipid levels. It has also been shown that activation of daf-16/FOXO can decrease the mRNA levels of a worm insulin gene, ins-7 (Murphy et al., Nature 424:277-283, 2003). This result is consistent with our results showing that DILP2 mRNA levels are decreased and reducing dTOR activity can reverse this dFOXO-mediated reduction of DILP2. This result suggests that reduction of dTOR function is increasing DILP2 levels to lower glucose levels, because the expression of activated dFOXO in the IPC leads to low DILP2 and elevated glucose levels, and the dTOR mutant can reverse both of these effects. These results are expected to parallel the role for FOXO and TOR in the regulation of insulin levels in mammals.

We also see a selective and unexpected regulation of dTOR effectors: loss of d4EBP protein and a mild effect on dS6K Ser389 phosphorylation. It has also been recently shown that d4EBP is a target of dFOXO in Drosophila (Junger et al., J. Biol. 2:20, 2003; Miron et al., Molec. Cell. Biol. 23:9117-9126, 2003; Miron et al., Nature Cell Biol. 3:596-601, 2001; Tettweiler et al., Genes Dev. 19:1840-1843, 2005) and thus may represent one of the dTOR targets responsible for blocking the dFOXO-mediated metabolic phenotypes. It has also been shown that daf-15/Raptor is a target of daf-16/FOXO in the worm and may also contribute to the dTOR metabolic and senescent phenotypes (Jia et al., Development 131:3897-3906, 2004). Raptor may also contribute to the selective difference in the regulation of d4EBP and dS6K function by TOR because Raptor binds to both S6K and 4EBP and loss of 4EBP may allow for more S6K binding to Raptor for TOR-mediated phosphorylation (Hara et al., Cell 110:177-189, 2002). Thus, these results suggest that reduction of dTOR function may have selective effects on translation.

Reduction of dTOR function does not provide resistance against acute stresses or cause sterility. This result is in contrast to the yeast TOR1 mutant, which shows elevated stress resistance (Powers et al., Genes Dev. 20:174-184, 2006), and the d4EBP mutant, which shows stress and starvation sensitivity (Bernal and Kimbrell, Proc. Natl. Acad. Sci. USA 97:6019-6024, 2000; Tettweiler et al., Genes Dev. 19:1840-1843, 2005). Nevertheless, the dTOR^{7/P} mutant has an increased lifespan. This result is in keeping with the yeast, worm and fly studies that show that loss of TOR signaling can increase lifespan, possibly as a caloric restriction mediator (Kaeberlein et al., Science 310:1193-1196, 2005; Kapahi et al., Curr. Biol. 14:885-890, 2004; Powers et al., Genes Dev. 20:174-184, 2006; Vellai et al., Nature 426:620, 2003). Additionally, elevation of AMPK activity in C. elegans is also able to increase lifespan (Apfeld et al., Genes Dev. 18:3004-3009, 2004). Furthermore, reduction of dTOR activity prevents age-dependent functional decline of heart performance. Thus, reduction of dTOR function may reallocate energy stores preferentially for the control of ‘long-term’ responses such as lifespan and organ maintenance. Importantly, there are many potential links between changes in energy homeostasis with alterations in aging and organ senescence (Curtis et al., Nature Rev. Drug Discovery 4:569-580, 2005; Kenyon, Cell 120:449-460, 2005; Wessells et al., Nature Genetics 36:1275-1281, 2004). It is not currently clear how dTOR is regulating these organ and organismal responses, but the altered lipid metabolism may underlie these changes. Funneling diverse stimuli like amino acids, growth factors, oxygen tension, and energy charge into the TOR pathway may be an economic method to mobilize fuel stores like lipids to counteract these fluctuations.

### CONCLUSIONS

The conservation of basic mechanisms between Drosophila and mammals is well established. It has been shown that disruption of insulin signalling in non-mammalian systems like Drosophila results in altered glucose and lipid levels (Broughton et al., Proc. Natl. Acad. Sci. USA 102:3105-3110, 2005; Rulifson et al., Science 296:1118-1120, 2002). Furthermore, human insulin can activate the Drosophila insulin receptor (Rulifson et al., Science 296:1118-1120, 2002). We found that reducing dTOR function can reverse activated dFOXO-mediated insulin resistance phenotypes induced in both insulin producing and insulin receiving tissues, and thus this study provides the first direct evidence that reducing TOR function may have a clinical benefit to counter insulin resistance, metabolic syndrome, and/or diabetes. Furthermore, altering TOR signaling may underlie the benefits of various diet and nutritional regimens. The TOR system evolved about 1 billion years before the insulin/IGF system became extant, and TOR can be viewed as an ancient “master” integrator of energy homeostasis. Thus, the basic mechanisms controlling glucose and lipid homeostasis, including mechanisms by which the TSC₁₋₂/TOR pathway influences insulin signaling as well as the influence of TSC₁₋₂/TOR signaling on peripheral tissue and IPC physiology, are also functionally conserved.

In unraveling the complex genetic network of TOR and 1nR signaling, the Drosophila model has been instrumental in finding critical components and uncovering functionally important genetic interactions between these two pathways (Oldham and Hafen, Trends Cell. Biol. 13:79-85, 2003). To conclude, we have described a new use for reducing dTOR activity to block insulin resistance, metabolic syndrome, and diabetic-like phenotypes downstream of activated dFOXO.

It is well established that the insulin/IGF and TOR pathways in Drosophila are functionally conserved with mammalian systems. We have shown that the corresponding allelic changes in mammalian TOR results in a partial decrease in kinase activity, which strongly argues that the effects are functionally conserved and that compounds that partially reduce mTOR activity/function will be applicable in humans.

All publications, patents and patent applications are incorporated herein by reference in their entirety. In case of conflict, the present specification, including definitions, will control. In addition, the materials, methods, and examples are illustrative only and not intended to be limiting.

While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

What is claimed is:

1. A composition comprising an amount of a TOR inhibitor that is effective for treating a condition in a patient selected from the group consisting of metabolic syndrome, insulin resistance, diabetes, obesity, cardiovascular disease, aging, and organ senescence.

2. The composition of claim 1 wherein the TOR inhibitor is effective in a rescue assay.

3. The composition of claim 1 comprising an amount of a TOR inhibitor that is effective for treating metabolic syndrome.

4. The composition of claim 1 comprising an amount of a TOR inhibitor that is effective for treating insulin resistance.

5. The composition of claim 1 comprising an amount of a TOR inhibitor that is effective for treating diabetes.

6. The composition of claim 1 comprising an amount of a TOR inhibitor that is effective for treating obesity.

7. The composition of claim 1 comprising an amount of a TOR inhibitor that is effective for treating cardiovascular disease.

8. The composition of claim 1 comprising an amount of a TOR inhibitor that is effective for treating aging.

9. The composition of claim 1 comprising an amount of a TOR inhibitor that is effective for treating organ senescence.

10. The composition of claim 1 wherein the TOR inhibitor reduces glucose levels in the patient.

11. The composition of claim 1 wherein the TOR inhibitor reduces lipid levels in the patient.

12. The composition of claim 1 wherein the TOR inhibitor reduces glucose and lipid levels in the patient.

13. The composition of claim 1 comprising a pharmaceutically acceptable carrier.

14. The composition of claim 1 further comprising an amount of an active ingredient other than the TOR inhibitor that is effective in treating the condition.

15. A composition comprising an amount of a TOR inhibitor that is effective in reducing glucose levels in a patient.

16. The composition of claim 15 wherein the TOR inhibitor is effective in reducing lipid levels in the patient.

17. The composition of claim 15 wherein the TOR inhibitor is effective in reducing glucose and lipid levels in the patient.

18. The composition of claim 15 wherein the TOR inhibitor is effective in a rescue assay.

19. The composition of claim 15 comprising a pharmaceutically acceptable carrier.

20. The composition of claim 15 further comprising an amount of an active ingredient other than the TOR inhibitor that is effective in reducing glucose levels in the patient.

21. A composition comprising an amount of a TOR inhibitor that is effective in reducing lipid levels in a patient.

22. The composition of claim 21 wherein the TOR inhibitor is effective in a rescue assay.

23. The composition of claim 21 comprising a pharmaceutically acceptable carrier.

24. The composition of claim 21 further comprising an amount of an active ingredient other than the TOR inhibitor that is effective in reducing lipid levels in the patient.

25. A composition comprising an amount of a TOR inhibitor that is effective in treating a condition in a patient characterized by abnormal FOXO activity.

26. The composition of claim 25 wherein the TOR inhibitor is effective in reducing glucose levels in the patient.

27. The composition of claim 25 wherein the TOR inhibitor is effective in reducing lipid levels in the patient.

28. The composition of claim 25 wherein the TOR inhibitor is effective in reducing glucose and lipid levels in the patient.

29. The composition of claim 25 wherein the TOR inhibitor is effective in a rescue assay.

30. The composition of claim 25 comprising a pharmaceutically acceptable carrier.

31. The composition of claim 25 further comprising an amount of an active ingredient other than the TOR inhibitor that is effective in treating the condition.

32. A method of treating a condition in a patient selected from the group consisting of metabolic syndrome, insulin resistance, diabetes, obesity, cardiovascular disease, aging, and organ senescence, the method comprising administering to the patient an effective amount of a composition comprising a TOR inhibitor.

33. The method of claim 32 wherein the TOR inhibitor is effective in a rescue assay.

34. The method of claim 32 wherein the condition is metabolic syndrome.

35. The method of claim 32 wherein the condition is insulin resistance.

36. The method of claim 32 wherein the condition is diabetes.

37. The method of claim 32 wherein the condition is obesity.

38. The method of claim 32 wherein the condition is cardiovascular disease.

39. The method of claim 32 wherein the condition is aging.

40. The method of claim 32 wherein the condition is organ senescence.

41. The method of claim 32 wherein the composition is effective in reducing glucose levels in the patient.

42. The method of claim 32 wherein the composition is effective in reducing lipid levels in the patient.

43. The method of claim 32 wherein the composition is effective in reducing glucose and lipid levels in the patient.

44. The method of claim 32 further comprising administering to the patient an active ingredient other than the TOR inhibitor that is effective in treating the condition.

45. The method of claim 44 wherein the composition comprises the active ingredient other than the TOR inhibitor.

46. A method of reducing glucose levels in a patient comprising administering to the patient an effective amount of a composition comprising a TOR inhibitor.

47. The method of claim 46 wherein the composition is effective in reducing lipid levels in the patient.

48. The method of claim 46 wherein the composition is effective in reducing glucose and lipid levels in the patient.

49. The method of claim 46 wherein the TOR inhibitor is effective in a rescue assay.

50. The method of claim 46 further comprising administering to the patient an active ingredient other than the TOR inhibitor that is effective in reducing glucose levels in the patient.

51. The method of claim 50 wherein the composition comprises the active ingredient other than the TOR inhibitor.

52. A method of reducing lipid levels in a patient comprising administering to the patient an effective amount of a composition comprising a TOR inhibitor.

53. The method of claim 52 wherein the TOR inhibitor is effective in a rescue assay.

54. The method of claim 52 further comprising administering to the patient an active ingredient other than the TOR inhibitor that is effective in reducing lipid levels in the patient.

55. The method of claim 54 wherein the composition comprises the active ingredient other than the TOR inhibitor.

56. A method of treating a condition in a patient characterized by abnormal FOXO activity composition, the method comprising administering to the patient an effective amount of a composition comprising a TOR inhibitor.

57. The method of claim 56 wherein the composition is effective in reducing glucose levels in the patient.

58. The method of claim 56 wherein the composition is effective in reducing lipid levels in the patient.

59. The method of claim 56 wherein the composition is effective in reducing glucose and lipid levels in the patient.

60. The method of claim 56 wherein the TOR inhibitor is effective in a rescue assay.

61. The method of claim 56 further comprising administering to the patient an active ingredient other than the TOR inhibitor that is effective in treating the condition.

62. The method of claim 61 wherein the composition comprises the active ingredient other than the TOR inhibitor.

63. A method of identifying a substance that is effective in treating a condition selected from the group consisting of metabolic disorder, insulin resistance, diabetes, obesity, cardiovascular disease, aging, and organ senescence, the method comprising: (a) determining whether the substance is a TOR inhibitor; and (b) determining whether the substance is effective in treating the condition.

64. The method of claim 63 wherein said step of determining whether the substance is a TOR inhibitor comprises providing a cell that has a mutation that confers a detectable phenotype in the presence of a TOR inhibitor; (b) contacting the cell with a composition comprising the substance; (c) determining whether the cell displays the phenotype upon when contacted with the composition.

65. The method of claim 63 wherein the mutation is in one or more genes selected from the group consisting of TSC2, TSC2, AMPK, and LKB.

66. The method of claim 63 wherein the mutation causes activation or overexpression of FOXO.

67. The method of claim 63 wherein the mutation causes overexpression of one or more proteins selected from the group consisting of Rheb, TOR or S6K.

68. The method of claim 63 wherein the cell is a Drosophila embryo, larva or adult.

69. The method of claim 63 further comprising determining whether the substance reduces lipid levels in a suitable assay.

70. The method of claim 63 further comprising determining whether the substance reduces glucose levels in a suitable assay.

71. The method of claim 63 further comprising determining whether the substance reduces lipid levels and glucose levels in a suitable assay.

72. A method of identifying a substance that is effective in treating a condition in a patient that is characterized by abnormal FOXO activity, the method comprising: (a) determining whether the substance is a TOR inhibitor; and (b) determining whether the substance is effective in treating the condition in the patient.

73. A method of identifying a substance that is effective in reducing glucose levels in a patient, the method comprising: (a) determining whether the substance is a TOR inhibitor; and (b) determining whether the substance is effective in reducing glucose levels in the patient.

74. The method of claim 73 further comprising determining whether the substance is effective in reducing lipid levels in a suitable assay.

75. A method of identifying a substance that is effective in reducing lipid levels in a patient, the method comprising: (a) determining whether the substance is a TOR inhibitor; and (b) determining whether the substance is effective in reducing lipid levels in the patient.


SEQUENCE LISTING (14 sequences)

SEQ ID NO: 1 - protein, 40 aa, Drosophila sp.
CIRGSNGKDYMYLLKGHEDLRQDERVMQLFSLVNTLLLDD

SEQ ID NO: 2 - protein, 40 aa, Homo sapiens
TLMGSNGHEFVFLLKGHEDLRQDERVMQLFGLVNTLLAND

SEQ ID NO: 3 - protein, 40 aa, Saccharomyces cerevisiae
SIKGSDGKDYKYVLKGHEDIRQDSLVMQLFGLVNTLLKND

SEQ ID NO: 4 - protein, 40 aa, Saccharomyces cerevisiae
CIKGSDGKDYKYVLKGHEDIRQDSLVMQLFGLVNTLLQND

SEQ ID NO: 5 - protein, 40 aa, Arabidopsis thaliana
TIHGNDGEDYAFLLKGHEDLRQDERVMQLFGLVNTLLENS

SEQ ID NO: 6 - protein, 40 aa, Caenorhabditis elegans
VIRGSNGLDYQFLLKGHEDPRQDERVMQLFGLVNTLLANN

SEQ ID NO: 7 - DNA, 16 bp, Artificial Sequence
  Description of Artificial Sequence Synthetic primer
actacacgacgtagcc

SEQ ID NO: 8 - DNA, 16 bp, Artificial Sequence
  Description of Artificial Sequence Synthetic primer
caacctcgggatcact

SEQ ID NO: 9 - DNA, 21 bp, Artificial Sequence
  Description of Artificial Sequence Synthetic primer
ctgggagctatcttgggggtg

SEQ ID NO: 10 - DNA, 20 bp, Artificial Sequence
  Description of Artificial Sequence Synthetic primer
cgcgtcgaccaggtcagttg

SEQ ID NO: 11 - DNA, 17 bp, Artificial Sequence
  Description of Artificial Sequence Synthetic primer
gacgaatgtgaccttgg

SEQ ID NO: 12 - DNA, 17 bp, Artificial Sequence
  Description of Artificial Sequence Synthetic primer
acccgagtattgggtga

SEQ ID NO: 13 - DNA, 20 bp, Artificial Sequence
  Description of Artificial Sequence Synthetic primer
taccccattgagcacggtat

SEQ ID NO: 14 - DNA, 20 bp, Artificial Sequence
  Description of Artificial Sequence Synthetic primer
ggtcatcttctcacggttgg